(19) United States
(12) Patent Application Publication
(10) Pub. No.: US 2026/0259601 A1
(43) Pub. Date: Sep. 3, 2026

(54) PUPIL DETECTION METHOD, PUPIL DETECTION DEVICE AND PAIR OF SMART GLASSES

(71) Applicant: Robert Bosch GmbH, Stuttgart (DE)

(72) Inventor: Johannes Meyer, Haseluenne (DE)

(21) Appl. No.: 19/164,740

(22) PCT Filed: Jun. 3, 2024
(86) PCT No.: PCT/EP2024/065208

§ 371 Date: Sep. 12, 2025

(30) Foreign Application Priority Data
Jun. 22, 2023 (DE) 10 2023 205 844.8

(51) Int. Cl. G06F 3/01

(52) U.S. Cl. CPC G06F 3/013

(57) ABSTRACT

A pupil detection method for a pair of smart glasses. The method includes: at least one scanning step, in which a laser beam, in particular an infrared laser beam, of at least one laser feedback interferometry sensor is scanned over at least a majority of a visible portion of an eye of a user, using a MEMS micromirror system; and at least one capturing step, in which a backscattered portion of the laser beam, backscattered by the eye of the user, is captured by the LFI sensor and converted into an electrical signal. In at least one recording step, signal spikes in the electrical signal which in particular exceed a definable threshold value are recorded together with scan coordinates, in particular of the MEMS micromirror system, which are associated with the respective signal spikes, in order to detect a pupil of the eye of the user.

### BACKGROUND INFORMATION

A pupil detection method for a pair of smart glasses, comprising at least one scanning step, in which a laser beam of a laser feedback interferometry (LFI) sensor is scanned over at least a majority of a visible portion of an eye of a user, and comprising at least one capturing step, in which a backscattered portion of the laser beam, said backscattered portion being backscattered by the eye of the user, is captured by the LFI sensor and converted into an electrical signal, has already been proposed. For example, an infrared laser beam scanned over the eye of the user is backscattered according to the reflectivity of the eye components of the eye of the user and, from the reflection signal, a two-dimensional image is created in which the pupil appears bright due to increased reflectivity of the retina (“bright pupil effect”). By means of an image recognition algorithm, such as a Canny edge detector, a bright pupil contour is recognized in the image and then a pupil ellipse is determined. This pupil detection method requires continuous sampling, which causes relatively high power consumption, in order capture the complete two-dimensional images.

### SUMMARY

The present invention proceeds from a pupil detection method, in particular for a pair of smart glasses, comprising at least one scanning step, in which a laser beam, in particular an infrared laser beam, of at least one laser feedback interferometry (LFI) sensor is scanned, in particular two-dimensionally, over at least a majority of a visible portion of an eye of a user, in particular by means of a MEMS micromirror system, and comprising at least one capturing step, in which a backscattered portion of the laser beam, said backscattered portion being backscattered by the eye of the user, is at least partially captured by the LFI sensor and converted, preferably with the aid of an electronic unit, into an electrical signal.

According to an example embodiment of the present invention, in at least one recording step, signal spikes in the electrical signal which in particular exceed a definable threshold value (limit value/threshold) are recorded, preferably exclusively, together with scan coordinates, in particular of the MEMS micromirror system, which are associated with the respective signal spikes, in order to detect a pupil of the eye of the user. As a result, efficiency can advantageously be increased. Advantageously, power consumption required for pupil detection can be significantly reduced. Advantageously, continuous sampling of photodiode signals, e.g., of the LFI sensor, can be dispensed with. In addition, a memory requirement for signals/images recorded by the LFI sensor can advantageously be significantly reduced, in particular because only individual signal spikes need to be recorded instead of entire continuous images. In addition, image processing algorithms can be advantageously dispensed with or at least power consumption of image processing algorithms can be substantially reduced, in particular since the signals/images to which these image processing algorithms are applied are substantially simpler and/or of smaller data size.

A “pair of smart glasses” is in particular to be understood as a wearable (head-mounted display), by means of which information can be added to the field of view of a user. Pairs of smart glasses preferably make augmented reality applications, virtual reality applications and/or mixed reality applications possible. Smart glasses are also commonly referred to as VR glasses or AR glasses. In particular, the pair of smart glasses comprises a virtual retinal scan display (also known as a light field display), in particular one generally conventional to a person skilled in the art. The virtual retinal scan display is in particular configured to scan image content sequentially by deflecting at least one visible laser beam from at least one time-modulated light source, such as one or more (RGB) laser diodes of a laser projector, and to image it directly onto the retina of the eye of the user by means of optical elements. The pupil detection method may in particular be used for eye tracking of an eye tracking system preferably integrated into the pair of smart glasses. An eye tracking system is in particular configured to track and/or record movement of at least the eye of the user. In particular, the LFI sensor forms a part, in particular an integral part, of the eye tracking system of the pair of smart glasses. The eye tracking system could be provided for recognizing an eye position and/or pupil position of the eye of the user by means of the so-called “dark pupil effect”, but preferably the eye tracking system is provided at least for recognizing the eye position and/or pupil position by means of the so-called “bright pupil effect”, which is based in particular on the relatively high infrared reflectivity of the retina of the eye in comparison with adjacent/other components of the eye of the user. The terms “configured” and “provided” are in particular understood to mean specifically programmed, designed, and/or equipped. An object being provided or configured for a particular function is in particular understood to mean that the object fulfills and/or performs this particular function in at least one application state and/or operating state.

According to an example embodiment of the present invention, the LFI sensor may be in the form of a VCSEL, preferably ViP-VCSEL (“vertical-cavity surface-emitting laser with integrated photodiode”), for example. The LFI sensor is in particular integrated into the pair of smart glasses, e.g., into a glasses frame, glasses lens or glasses temple of the pair of smart glasses. The LFI sensor may be integrated into a common laser module/a common laser projector with the (RGB) laser diodes of the pair of smart glasses which generate the projected image. Alternatively, the LFI sensor may also be separate from the laser projector with the (RGB) laser diodes which generate the projected image. It is also possible that the pair of smart glasses comprises more than one LFI sensor, e.g., two, three or more than three LFI sensors. The LFI sensor is based on an interferometric measurement method. The LFI sensor may additionally be able to capture a distance to a target (e.g., the eye of the user) as well as a surface velocity of the target. In particular, the LFI sensor emits the laser beam in the infrared spectrum, and the laser beam then hits, at an angle Y, a surface having a reflectivity R. From this surface, the light of the laser beam is then backscattered such that it enters a laser cavity of the LFI sensor again. In the laser cavity of the LFI sensor, the backscattered light interferes with a locally oscillating field of the LFI sensor. This leads in particular to modulation of laser power of the laser source, which may be captured selectively either by a photodiode integrated into a rear reflector of the laser cavity or by a direct measurement of a voltage of the laser source. In particular, the LFI sensor photodiode integrated into the laser module is provided for capturing the backscattered portion of the laser beam and converting said backscattered portion into the electrical signal. Furthermore, a current of the laser source may be modulated with a preferably triangular modulation signal in order to achieve a cyclic shift of the wavelength of the laser. If the laser parameters are known, a beat frequency and a Doppler frequency may subsequently be determined. From these quantities, the surface velocity of the eye, the distance of the LFI sensor from the eye and other eye parameters can be ascertained using conventional equations.

According to an example embodiment of the present invention, the MEMS micromirror system preferably forms at least a portion of a microscanner. The microscanner is, in particular, a micro-opto-electro-mechanical system of the class of micromirror actuators for dynamic modulation of light. A modulation-causing movement of individual mirrors of the MEMS micromirror system may be performed rotationally at least about one or about two axes, whereby in particular deflection of light beams incident on the individual mirrors occurs. In particular, the scanned laser beam is scanned row by row. However, scanning the scanned laser beam column by column is also possible. Typical dimensions of individual mirrors of a MEMS micromirror system lie in the range between one and three millimeters. However, larger mirror apertures (e.g., 10 mm×3 mm) can also be produced. Scanning frequencies of the MEMS micromirror system lie in the range of several kilohertz, in particular in the range of up to 50 KHz.

The visible portion of the eye of the user, over which visible portion the laser beam is to be scanned, is formed at least predominantly by the pupil of the eye of the user, an iris of the eye of the user, and a sclera of the eye of the user. In particular, the retina produces a so-called speckle pattern upon reflection of a laser beam, in particular a sufficiently coherent laser beam. The speckle pattern (also known as laser granulation or light granulation) is in particular an interference phenomenon which has a granular appearance and which develops in the event of sufficiently coherent illumination of optically rough object surfaces, such as the retina of the eye (cf. cones and rods of the retina). In particular, the individual speckles of the speckle pattern are captured by the photodiode of the LFI sensor. In particular, individual speckles of the speckle pattern that hit the photodiode of the LFI sensor each generate at least one signal spike of the electrical signal there. In particular, speckles of the speckle pattern which is generated by the retina by (back) reflection are recorded in the recording step.

In particular, according to an example embodiment of the present invention, the pair of smart glasses comprises an electronic unit and/or a computing unit, which is provided at least for receiving the electrical signal of the capturing step and/or recording at least a portion of the electrical signal of the capturing step, in particular the signal spikes. Preferably, the recorded signal spikes are linked, preferably saved with linking, to the respective scan coordinates that were current at the time of measurement by the electronic unit and/or by the computing unit. Preferably, in the pupil detection method only the signal spikes are recorded, while in particular all further parts of the electrical signal remain unconsidered and/or even unrecorded. In particular, the threshold value which determines whether a signal is recorded or not is definable. Preferably, the electrical signal of an event to be identified as a signal spike is at least twice as high, advantageously at least three times as high, preferably at least five times as high, and particularly preferably at least ten times as high as the electrical signal immediately before and immediately after the event and/or as a mean value or noise of the electrical signal. The term “computing unit” is in particular understood to mean a unit with an information input, information processing, and an information output. Advantageously, the computing unit comprises at least one processor, a memory, input and output means, further electrical components, an operating program, regulating routines, control routines, and/or calculation routines. The components of the computing unit are preferably arranged on a common printed circuit board and/or are advantageously arranged in a common housing.

According to an example embodiment of the present invention, it is also provided that, in at least one contour ascertainment step, at least a portion of a pupil contour of the pupil of the eye of the user is ascertained from a plurality of first signal spikes in different scan rows of the laser beam which is scanned, in particular by means of the MEMS micromirror system, and/or from a plurality of last signal spikes in different scan rows of the laser beam which is scanned, in particular by means of the MEMS micromirror system. In this way, particularly efficient pupil detection, in particular pupil contour capturing, can advantageously be made possible. Advantageously, a necessary data volume and/or a necessary power consumption for pupil detection can be kept low. In addition, an increase in a spatial (two-dimensional) resolution of the pupil contour can advantageously be achieved in this way, in particular since an analog (quasi-direct) detection of the pupil edge can be made possible. In addition, direct (“on-the-fly”) capturing of the pupil contour and/or a pupil center, e.g., by means of (adaptive) fitting, during the ongoing image capturing/the ongoing scanning step/scanning of the MEMS micromirror system could advantageously be made possible. In this way, system latency can be advantageously minimized. Advantageously, pupil parameters such as the pupil contour and/or the pupil center, etc., can thereby already be provided immediately at the end of each scanning process. In particular, LFI sensors have comparatively little susceptibility to disturbing light, and thus the number of false triggerings of the recording is low/particularly few false triggerings of the recording occur. A first signal spike represents in particular a first instance of the definable threshold value being exceeded by the electrical signal of the photodiode of the LFI sensor in a scan row or scan column of a scan scheme of the MEMS micromirror system. A last signal spike represents in particular a last instance of the definable threshold value being exceeded by the electrical signal of the photodiode of the LFI sensor in a scan row or scan column of a scan scheme of the MEMS micromirror system. In particular, in the scanning region overlapping with the retina, each scan row comprises a first signal spike and a last signal spike. Further signal spikes can be registered/can lie between the first signal spike and the last signal spike of a scan row. The remaining signal spikes of a scan row between the first signal spike of the scan row and the last signal spike of the scan row, in particular the further signal spikes of the scan row, may be filtered out and/or discarded. The pupil contour represents in particular an outer outline of the pupil in the scan coordinates, in particular in a coordinate system of the pair of smart glasses.

Furthermore, according to an example embodiment of the present invention, it is provided that, in at least one pupil ascertainment step, a pupil shape and/or a pupil position of the pupil of the eye of the user is ascertained from a merely partial, in particular approximately half, pupil contour or from a full-periphery pupil contour, for example by means of an ellipse fitting algorithm (e.g., least squares) of the computing unit and/or of the electronic unit. In this way, fast and/or energy-saving pupil ascertainment can advantageously be made possible. If a complete pupil contour and/or pupil shape of the eye of the user and in particular their relationship to the coordinate system of the pair of smart glasses is known, a gaze vector of the eye of the user can be ascertained by means of an conventional algorithm from the related art, such as a Swirski algorithm. The partial, e.g., half, pupil contour can be ascertained, for example, from an upper or lower half with at least two captured signal spikes per scan row. The partial, e.g., half, pupil contour can be ascertained, for example, from a lateral half with only one signal spike, in particular only one first signal spike or only one last signal spike, per row.

According to an example embodiment of the present invention, it is also proposed that the signal spikes recorded in the recording step correspond to (individual) speckles, in particular of the speckle pattern, which are generated by (back) scattering of the laser beam, which is applied in the scanning step, at a retina of the eye of the user. This advantageously makes it possible to achieve reliable recognition of the pupil by utilizing a property of the retina. In particular, the speckles, which are in the form of bright light spots, generate the signal spikes in the electrical signal of the photodiode of the LFI sensor upon re-entry into the LFI sensor.

In addition, according to an example embodiment of the present invention, it is provided that, in the recording step, the electrical signal is monitored, in particular by means of a Schmitt trigger, with respect to whether a threshold value is exceeded, and an interrupt is triggered if the threshold value is exceeded, the interrupt triggering a query of the current scan coordinates, in particular of the position of the micromirror system assumed at the time point of the interrupt, preferably by means of a digital processor. In this way, speckles generated by the retina can advantageously be safely and/or reliably associated with scan coordinates, in particular in the coordinate system of the pair of smart glasses. The scan coordinates are preferably stored together with a Schmitt trigger binary signal in a memory of the pair of smart glasses, in particular in the form (α, β, binary variable). α and β are preferably angles/angular positions, in particular current pivoting angles/current pivoting angular positions, of the MEMS micromirror system, in particular of the relevant individual mirror of the MEMS micromirror system. The binary variable is preferably a binary signal which is associated with the Schmitt trigger and which, for example, can be 0 if no threshold value is exceeded and 1 if a threshold value is exceeded, or vice versa.

If, in at least one training step, the threshold value is iteratively adjusted, in particular with each new frame generated by the scanned laser beam, preferably until the number of signal spikes per frame falls below a definable maximum number, e.g., 10 or 20 or 30, a particularly well-optimized balance of sensitivity and power consumption can advantageously be obtained. Advantageously, high efficiency can be achieved.

Preferably, the threshold values, in particular of the Schmitt trigger, are set digitally by means of a control signal. In particular, the threshold value, e.g., of the Schmitt trigger, can be set one time for a given user in the training step, in particular because the threshold value advantageously does not change by virtue of insensitivity of the LFI sensor to ambient light. The iterative adjustment of the threshold value can be carried out in an automated way, e.g., by means of the computing unit and/or the electronic unit, or with at least partially monitoring, e.g., by means of a confirmation of the user of the pair of smart glasses. Alternatively, to set the threshold value of the Schmitt trigger, the Schmitt trigger may be bridged during the training step and the complete signal (back-reflection signal) recorded for an entire frame (full scan of the MEMS micromirror system), the threshold value subsequently being defined on the basis of the (e.g., 5% or 10%) highest electrical signals, in particular voltages. The highest electrical signals, in particular voltages, preferably correspond to the brightest speckles/amplitudes of the measured back-reflected portion of the scanned laser beam. In particular, the Schmitt trigger at least partially forms the electronic unit and/or the computing unit. Alternatively, the electronic unit and/or the computing unit may also comprise other programmable threshold detectors for electrical signals, in particular voltage signals, which preferably perform identical tasks to those described above, by way of example, for the Schmitt trigger. As soon it is determined that the threshold value is exceeded, the threshold detector can trigger an interrupt in a connected digital processor of the electronic unit or of the computing unit.

In addition, according to an example embodiment of the present invention, it is provided that, in the scanning step, the laser beam is scanned in alternating directions, in particular sinusoidally, at least over the majority of the visible portion of the eye of the user. This may advantageously allow the pupil contour to be ascertained directly from an analog signal even without filtering out and/or discarding events (signal spikes). Advantageously, power consumption and/or a data rate/a data volume can be further reduced as a result. In particular, in this case only each first signal spike is required in order to be able to ascertain the pupil contour directly from the analog signal. Advantageously, ellipse fitting algorithms may be dispensed with. The alternate scanning may begin from the left (“even line”) or from the right (“odd line”). From the “even lines”, in particular from the scan rows running from left to right, a left pupil edge of the pupil of the eye of the user can thus be ascertained. From the “odd lines”, in particular from the scan rows running from right to left, a right pupil edge of the pupil of the eye of the user can thus be ascertained. For this purpose, the electronic unit, preferably the Schmitt trigger, may comprise an interruption module for an output signal of the Schmitt trigger, said interruption module being provided for opening an output of the Schmitt trigger after a first triggering of the Schmitt trigger as a result of the threshold value being exceeded, so that preferably no further events can be ascertained for the current scan row. The interruption by the interruption module may then be reset for each new row, e.g., by an h sync signal of the MEMS micromirror system. The term “majority” is understood to mean in particular 51%, desirably 66%, advantageously 75%, preferably 85%, and particularly preferably 95%.

If at least one electronic means, in particular of the electronic unit, which is provided for capturing the signal spikes in the electrical signal, for example a Schmitt trigger and/or a photodiode of the LFI sensor, is deactivated, after a first signal spike has been captured in a scan row of the laser beam scanned in alternating directions, for the remainder of the scan row, e.g., by the interruption module, and in particular is reset and/or reactivated only once a transition to a subsequent scan row occurs, particularly high energy efficiency/particularly low power consumption can advantageously be achieved.

In addition, according to an example embodiment of the present invention, it is provided that the electrical signal is a voltage signal and, in the conversion of the portion of the laser beam that is backscattered into the LFI sensor into the electrical signal in the capturing step, a photocurrent of a photodiode of the LFI sensor is converted into a voltage signal proportional to the photocurrent, in particular by means of a transimpedance amplifier of the electronic unit. In this way, simple and reliable detection of signal spikes can advantageously be made possible. A simple design can advantageously be made possible.

According to an example embodiment of the present invention, a pupil detection device, in particular for a pair of smart glasses, comprising at least one projector unit, which comprises at least one laser feedback interferometry (LFI) sensor and which is configured at least to scan a laser beam, in particular an infrared laser beam, over at least a majority of a visible portion of an eye of a user, in particular by means of a MEMS micromirror system of the projector unit, and which is configured at least to capture, at least partially, a backscattered portion of the laser beam, said backscattered portion being backscattered by the eye of the user, by means of the LFI sensor and to convert said backscattered portion into an electrical signal, in particular with the aid of the electronic unit, and comprising the electronic unit, which is configured at least to recognize signal spikes in the electrical signal which in particular exceed a definable threshold value and to record said signal spikes together with scan coordinates, in particular of the MEMS micromirror system, which are associated with the respective signal spikes, in order detect a pupil of the eye of the user, is also provided, as well as a pair of smart glasses comprising the pupil detection device. As a result, efficiency can advantageously be increased. Advantageously, power consumption required for pupil detection can be significantly reduced.

The pupil detection method according to the present invention, the pupil detection device according to the present invention and the pair of smart glasses according to the present invention are not to be limited to the application and embodiment described above. In particular, for fulfilling a functionality described here, the pupil detection method according to the invention, the pupil detection device according to the invention and the pair of smart glasses according to the invention can comprise a number of individual elements, components, units, and method steps that deviates from a number mentioned here. In addition, for the value ranges specified in this disclosure, values within the mentioned limits are also to be considered disclosed and usable as desired.

### BRIEF DESCRIPTION OF THE DRAWINGS

Further advantages result from the following description of the figures. Embodiment examples of the present invention are illustrated in the figures. The disclosure contains numerous features in combination. A person skilled in the art will expediently also consider the features individually and combine them to form meaningful further combinations.

FIG. 1 shows a schematic illustration of a portion of a pair of smart glasses having a pupil detection device for performing a pupil detection method, according to an example embodiment of the present invention.

FIG. 2 shows a schematic circuit-diagram illustration with a projector unit of the pupil detection device and with an electronic unit of the pupil detection device, according to an example embodiment of the present invention.

FIG. 3 shows a detailed view of a portion of the electronic unit, shown schematically, according to an example embodiment of the present invention.

FIG. 4 shows a schematic flowchart of the pupil detection method, according to an example embodiment of the present invention.

FIG. 5 shows three variables plotted against a common time: an h sync signal of a MEMS micromirror system of the pupil detection device (top), a v sync signal of the MEMS micromirror system (middle), and a binary signal which is ascertained from an electrical signal of the electronic unit and which indicates signal spikes (bottom).

FIG. 6 shows a frame generated by the projector unit, with the signal spikes registered in this frame at certain scan coordinate pairs, according to an example embodiment of the present invention.

FIG. 7 shows the same frame as in FIG. 6 with filtering of the signal spikes, according to the present invention.

FIG. 8 shows an example in which only first and last signal spikes of a subset of all scan rows of a frame were ascertained, according to the present invention.

FIG. 9 shows a scan pattern, shown schematically, of the MEMS micromirror system, in which scan rows are scanned in opposite directions in alternation.

FIG. 10 shows a schematic indication of the scan rows in the frame which was created by means of the scan pattern running in opposite directions in alternation.

### DETAILED DESCRIPTION OF EXAMPLE EMBODIMENTS

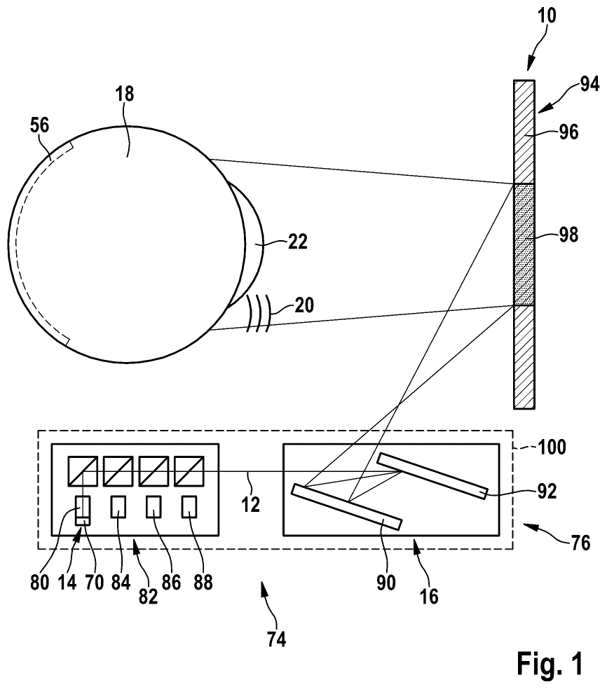

FIG. 1 shows a pair of smart glasses 10 schematically. The pair of smart glasses 10 comprises a virtual retinal scan display 94. The pair of smart glasses 10 comprises at least one glasses lens 96. The virtual retinal scan display 94 comprises a combiner 98. The combiner 98 is in the form of a holographic optical element (HOE), for example. The combiner 98 is provided for introducing an image generated by a projector unit 76 of the pair of smart glasses 10 into a field of view of a user of the pair of smart glasses 10. In the illustrated case, the combiner 98 is integrated into the glasses lens 96.

The pair of smart glasses 10 is provided for performing a pupil detection method. The pair of smart glasses 10 comprises a pupil detection device 74. The pupil detection device 74 comprises the projector unit 76. The pair of smart glasses 10 comprises a glasses frame 100. The projector unit 76 is at least partially integrated into the glasses frame 100. The projector unit 76 comprises at least one laser feedback interferometry (LFI) sensor 14. The LFI sensor 14 is provided for generating and outputting a laser beam 12. The laser beam 12 is coherent. The laser beam 12 is an infrared laser beam. The laser beam 12 is provided at least for pupil detection of a pupil 22 of an eye 18 of the user of the pair of smart glasses 10. The LFI sensor 14 comprises at least one infrared laser diode 80 for generating the laser beam 12. The LFI sensor 14 comprises at least one photodiode 70 for detecting and/or capturing at least infrared light, preferably at least a portion of a backscattered portion 20 of the laser beam 12, said backscattered portion being backscattered by the eye 18 of the user. The LFI sensor 14 is, for example, part of a laser module 82 of the projector unit 76, said laser module additionally comprising further laser diodes 84, 86, 88 for generating and outputting visible laser light.

The further laser diodes 84, 86, 88 form an RGB laser system. The further laser diodes 84, 86, 88 are each provided for outputting visible red laser beams, visible green laser beams, or visible blue laser beams. The further laser diodes 84, 86, 88 are provided for generating/projecting an image, e.g., of an augmented reality object, into the field of view of the user.

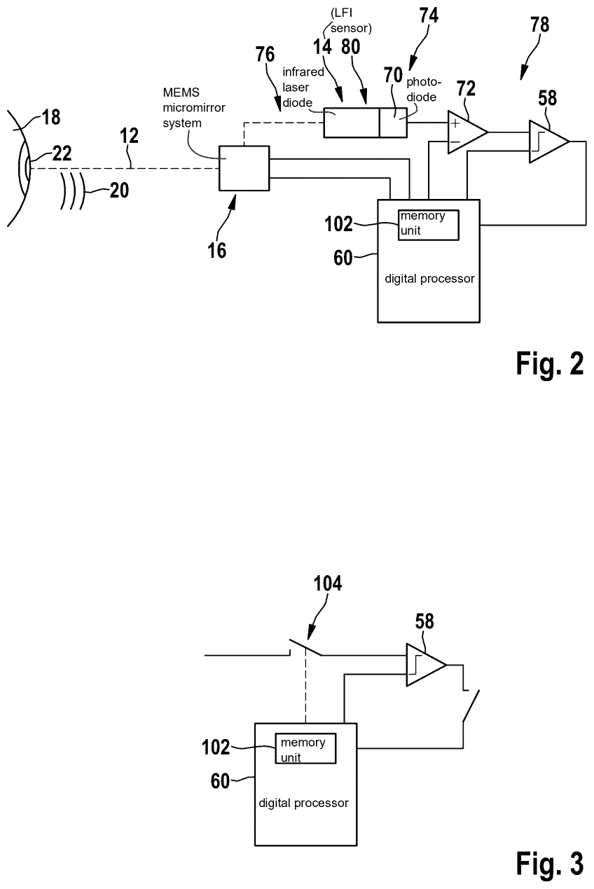

The projector unit 76 is provided for scanning the laser beam 12 at least over a majority of a visible portion of the eye 18 of the user. The projector unit 76 comprises a MEMS micromirror system 16. The MEMS micromirror system 16 comprises at least one movable, preferably pivotable, individual mirror 90. Preferably, the MEMS micromirror system 16 comprises at least two individual mirrors 90, 92 which can be pivoted orthogonally with respect to each other. The portion 20 of the laser beam 12 backscattered into the LFI sensor 14 generates a photocurrent in the photodiode 70. The pupil detection device 74, in particular the projector unit 76, comprises an electronic unit 78. The pupil detection device 74, in particular the projector unit 76, is provided for converting the captured backscattered portion 20 of the laser beam 12 into an electrical signal, in particular with the aid of the electronic unit 78. The electrical signal is a voltage signal. The pupil detection device 74, in particular the projector unit 76, is provided for converting the photocurrent into the electrical signal, in particular with the aid of the electronic unit 78, preferably with the aid of a transimpedance amplifier 72 (cf. FIG. 2) of the electronic unit 78. The electrical signal, which is in the form of a voltage signal, is proportional to the intensity of the generated photocurrent. The electronic unit 78 is configured at least to recognize signal spikes 24, 50, 52 (cf. inter alia FIG. 6) in the electrical signal and to record said signal spikes together with scan coordinates α, β (cf. inter alia FIG. 6) of the MEMS micromirror system 16 which are associated with the respective signal spikes 24, 50, 52, in order detect the pupil 22 of the eye 18 of the user. The electronic unit 78 is configured to record only the signal spikes 24, 50, 52 that exceed a definable threshold value.

FIG. 2 shows a schematic circuit-diagram illustration of the projector unit 76 and the electronic unit 78. The LFI sensor 14 with the integrated photodiode 70 emits the infrared laser beam 12 at a known wavelength towards the eye 18 of the user. This laser beam 12 is deflected two-dimensionally by the MEMS micromirror system 16. For deflection of the laser beam 12, deflection units other than a MEMS micromirror system 16 are also possible. The deflected laser beam 12 reaches the eye 18 of the user and is backscattered by the eye 18 of the user. A portion of the backscattered laser beam 12 is backscattered by a retina 56 of the eye 18 of the user and generates a speckle pattern due to the surface characteristics of the retina 56. The backscattered light of the laser beam 12 entering the LFI sensor 14 generates interference with a locally oscillating field within the LFI sensor 14. Positive interference results in an increase in the optical power of the laser, which is captured in the form of photocurrent by the integrated photodiode 70 of the LFI sensor 14. The transimpedance amplifier 72 amplifies the photocurrent signal and converts it into the corresponding proportional voltage signal. This measurement method is sensitive only to the self-emitted light and can therefore be performed independently of optical filters (e.g., laser line filters) or electronic filters (e.g., band pass filters).

The electronic unit 78 comprises a Schmitt trigger 58. The Schmitt trigger 58 is provided for monitoring analog signals, e.g., the electronic signal in the form of a voltage signal, with respect to whether a threshold value is exceeded. Alternatively, the electronic unit 78 could also have another programmable threshold detector. The voltage signal generated by the transimpedance amplifier 72 is fed to the Schmitt trigger 58. The electronic unit 78 comprises a digital processor 60 or is connected to a digital processor 60 of a computing unit of the pair of smart glasses 10. If it is determined that the threshold value is exceeded, an interrupt is triggered in the digital processor 60. The digital processor 60 is connected to the MEMS micromirror system 16. Upon occurrence of the interrupt, the digital processor 60 queries the current position of the individual mirror(s) 90, 92 of the MEMS micromirror system 16. The electronic unit 78 comprises a memory unit 102 or is connected to a memory unit 102 of the computing unit of the pair of smart glasses 10. The digital processor 60 is provided for storing, in the memory unit 102, the scan coordinates α, β obtained by the query triggered by the interrupt, as a detected event/as a speckle.

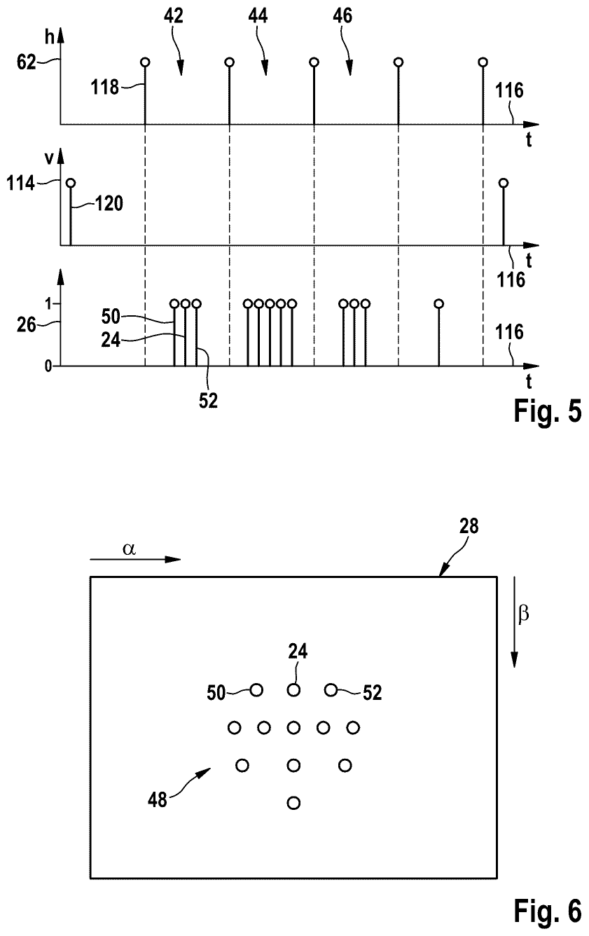

FIG. 3 shows a detailed view of a portion of electronics unit 78. The electronic unit 78 comprises an interruption switch 104. The interruption switch 104 is arranged at an input of the Schmitt trigger 58. The interruption switch 104 is in the closed state at the beginning of each scan row 42, 44, 46 (cf. FIG. 10) and is opened, e.g., by triggering by the digital processor 60, after the threshold value is exceeded for the first time, so that no further events can be detected for this scan row 42, 44, 46. Then, after the end of the current scan row 42, 44, 46 and before the start of the next scan row 42, 44, 46, the interruption switch 104 is reset, i.e., closed. This can be accomplished, for example, by receipt of an h sync signal 62 (cf. FIG. 5) from the MEMS micromirror system 16.

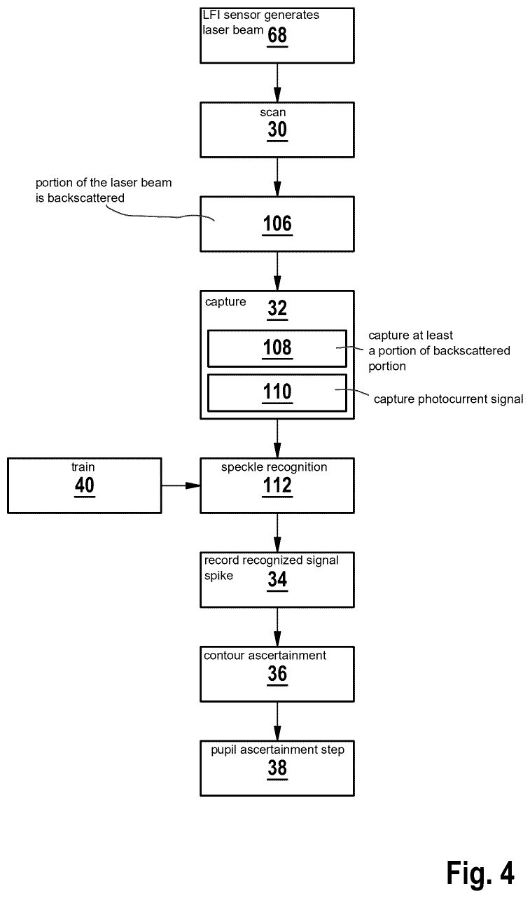

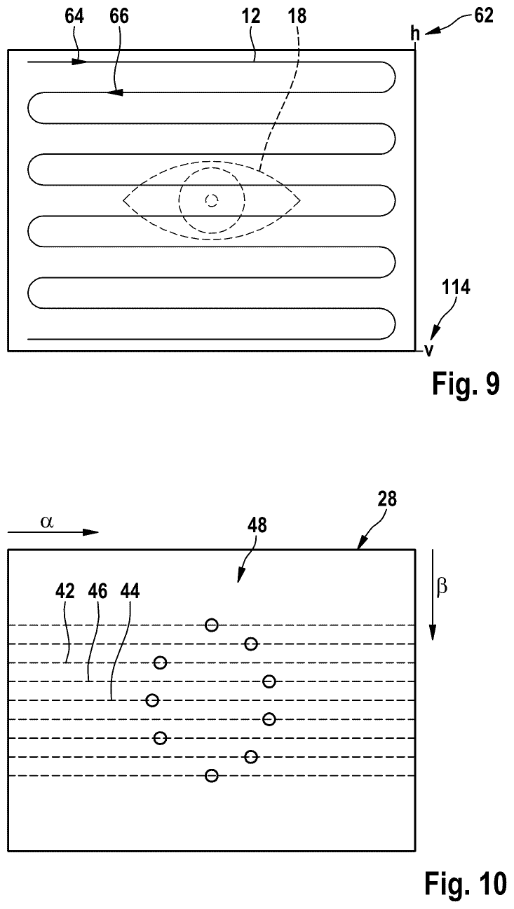

FIG. 4 shows a schematic flowchart of the pupil detection method. In at least one method step 68, the LFI sensor 14 generates the laser beam 12. In at least one scanning step 30, the laser beam 12 of the LFI sensor 14 is scanned over at least a majority of the visible portion of the eye 18 of the user by means of the MEMS micromirror system 16. The laser beam 12 can always be scanned in the same direction (always from right to left or always from left to right) in the scanning step 30. However, the laser beam 12 can also be scanned in alternate directions 64, 66, e.g., sinusoidally, in the scanning step 30 (cf. FIG. 9). In at least one method step 106, a portion 20 of the laser beam 12 is backscattered by the eye 18 of the user. Different regions of the eye 18 of the user generate different backscatterings. The retina 56 of the eye 18 of the user generates a speckle pattern when it reflects the coherent laser beam 12 back. In at least one sub-step 108 of a capturing step 32, at least a portion of the backscattered portion 20 of the laser beam 12, said backscattered portion being backscattered by the eye 18 of the user, is captured by the photodiode 70 of the LFI sensor 14. In at least one sub-step 110 of the capturing step 32, a photocurrent signal of the photodiode 70 is converted into the electrical signal. In at least one speckle recognition step 112, speckles of the speckle pattern generated by the retina 56 are recognized. The speckles generate respective signal spikes 24, 50, 52 in the electrical signal. By monitoring a definable threshold value, the signal spikes 24, 50, 52 are separated from the remainder of the signal of the photodiode 70 and identified as speckles. The threshold value is defined in a training step 40, which in particular is initially performed. This can be done manually or by automation. In the case of the automated performance of the training step 40, the threshold value is iteratively adjusted with each new frame 28 (cf. inter alia FIG. 6) generated by the scanned laser beam 12. This occurs until the number of signal spikes 24, 50, 52 per frame 28 falls below a definable maximum number, e.g., 10 or 20 or 30.

In at least one recording step 34, the recognized signal spikes 24, 50, 52 in the electrical signal which exceed the threshold value are recorded together with scan coordinates α, β of the MEMS micromirror system 16 which are associated with the respective signal spikes 24, 50, 52. The signal spikes 24, 50, 52 recorded in the recording step 34 thus correspond to speckles of the speckle pattern which is generated by the backscattering of the laser beam 12, which is applied in the scanning step 30, by the retina 56 of the eye 18 of the user. In the recording step 34, the electrical signal is monitored by means of the Schmitt trigger 58 with respect to whether the electrical signal exceeds the threshold value. When the threshold value is exceeded, the interrupt is triggered by the Schmitt trigger 58. The triggering of the interrupt in turn triggers a querying, by the digital processor 60, of the current scan coordinates α, β of the position of the micromirror system 16 assumed at the time point of the interrupt.

In at least one contour ascertainment step 36, at least a portion of a pupil contour 48, 54 of the pupil 22 of the eye 18 of the user is ascertained from the signal spikes 24, 50, 52. Merely a partial, e.g., half, pupil contour 54 (cf. FIG. 8) or a full-periphery pupil contour 48 (cf. FIG. 6) may be ascertained from the signal spikes 24, 50, 52. The partial pupil contour 54 may be ascertained from a plurality of first signal spikes 50 in different scan rows 42, 44 of the scanned laser beam 12. The partial pupil contour 54 may be ascertained from a plurality of last signal spikes 52 in different scan rows 42, 46 of the scanned laser beam 12. The full-periphery pupil contour 54 may be determined from the respective first signal spikes 50 and the respective last signal spikes 52 in different scan rows 42, 46 of the scanned laser beam 12. In at least one pupil ascertainment step 38, a pupil shape 124 and/or a pupil position of the pupil 22 of the eye 18 of the user is then ascertained from the merely partial, in particular approximately half, pupil contour 54 or from the full-periphery pupil contour 48. However, it is also possible that the full-periphery pupil contour 48 is determined only by means of first signal spikes 50. For this purpose, each successive scan row 42, 44, 46 is scanned in a different direction. In order to restrict the captured and stored signal spikes 24, 50, 52 to only the first signal spikes 50, electronics (cf. FIG. 3) are used, by means of which, after a first signal spike 50 has been captured in a scan row 42, 44, 46 of the scanned laser beam 12, the Schmitt trigger 58 is deactivated for the remainder of scan row 42, 44, 46 and is reset and reactivated only once a transition to a subsequent scan row 42, 44, 46 occurs. The described pupil detection method may also be used for a stereo approach, in particular by having multiple pupils 22 arranged in the scanning region of the LFI sensor 14 or when using stereoholograms as combiner 98.

FIG. 5 shows three variables plotted against a common time 116: the h sync signal 62 of the MEMS micromirror system 16 (top), a v sync signal 114 of the MEMS micromirror system 16 (middle), and a binary signal 26 ascertained from the electrical signal (bottom). The h sync signal 62 describes the reaching of a maximum/minimum horizontal scanning region by the MEMS micromirror system 16. A signal spike 118 of h sync signal 62 thus marks a start of a new scan row 42, 44, 46 in a frame 28. The v sync signal 114 describes the reaching a last scan row of a frame 28. The v sync signal 114 describes the maximum deflection angle of the MEMS micromirror system 16 in the vertical direction. A signal spike 120 of the v sync signal 114 thus marks a start of a new frame 28. The binary signal 26 describes the ascertained and stored signal spikes 24, 50, 52 of the electrical signal. If the electrical signal is below the threshold value, the binary signal 26 assumes the value 0. If the electrical signal exceeds the threshold value, the binary signal 26 assumes the value 1. The binary signal 26 thus shows the triggerings of the Schmitt trigger 58 by speckles of the speckle pattern and thus time points at which the pupil 22 of the eye 18 of the user was hit by the laser beam 12.

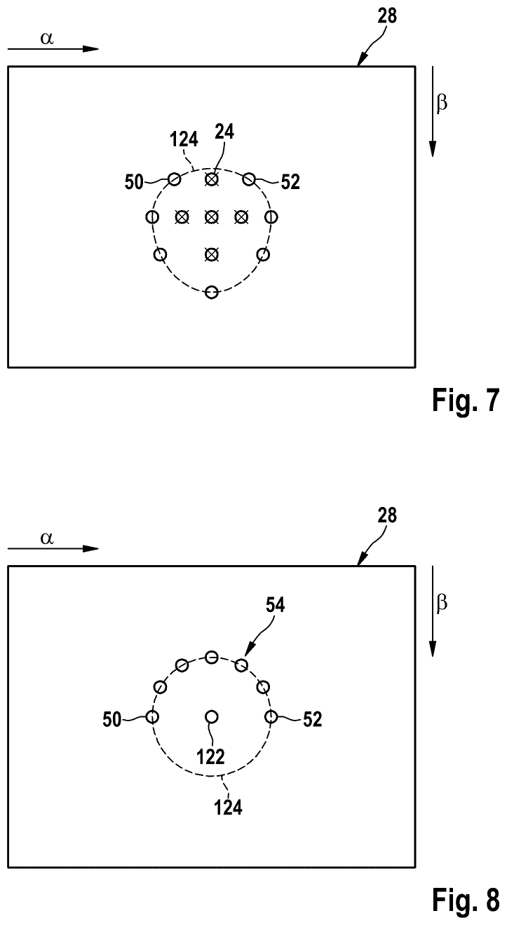

FIG. 6 shows a frame 28 generated by the projector unit 76, with the scan coordinates α, β and with the signal spikes 24, 50, 52 registered in this frame 28 at certain scan coordinate pairs. From the h sync signals 62 and the v sync signals 114 and from the time points of the occurrence of the signal spikes 24, 50, 52 with respect to the current h sync and v sync signals 62, 114, a current scan position of the MEMS micromirror system 16 in the scan coordinates α, β is determined and in particular is presented two-dimensionally in the frame 28. FIG. 7 shows the same measurement/the same frame 28 as FIG. 6; however, all signal spikes 24 that are not first signal spikes 50 of a scan row 42, 44, 46 or last signal spikes 52 of a scan row 42, 44, 46 have been filtered out and/or discarded. When the scan of the MEMS micromirror system 16 is performed row by row, the signal spikes 24, 50, 52 can be filtered within a scan row 42, 44, 46 so that, as in the illustrated case, only the first signal spikes 50 and the last signal spikes 52 in each scan row 42, 44, 46 are retained. All signal spikes 24 between them can be discarded without problems. On the basis of the remaining signal spikes 50, 52, the full-periphery pupil contour 54 can then be ascertained. FIG. 8 shows an example in which only the first and last signal spikes 50, 52 of a subset of all scan rows 42, 44, 46 of a frame 28 have been determined. A partial pupillary contour 48 is then ascertained from these signal spikes 50, 52. An ellipse fitting algorithm is then used to ascertain a center 122 of the pupil 22 in the scan coordinates α, β and/or a pupil shape 124 in the scan coordinates α, β.

FIG. 9 schematically shows a scan pattern of the MEMS micromirror system 16, in which the scan rows 42, 44, 46 are scanned in opposite directions 64, 66 in alternation. The scan pattern shown in FIG. 9 is sinusoidal. However, alternative scan patterns, e.g., rectangular scan patterns, are also possible. In FIG. 10, the scan rows 42, 44, 46 of a frame 28 which was created by means of such a scan pattern running in opposite directions in alternation are indicated schematically. In this case, recording only first signal spikes 50 is sufficient to allow the full-periphery pupil contour 48 to be ascertained. A left edge of the pupil 22 is ascertained from scan rows 42 that run from left to right (“even lines”). A right edge of the pupil 22 is ascertained from scan rows 44 that run from right to left (“odd lines”).

1-11. (canceled)

12. A pupil detection method for a pair of smart glasses, comprising the following steps:
in at least one scanning step, scanning an infrared laser beam of at least one laser feedback interferometry (LFI) sensor over at least a majority of a visible portion of an eye of a user, using a in particular by means of a microelectromechanical system (MEMS) micromirror system;
in at least one capturing step, at least partially capturing a backscattered portion of the laser beam using the LFI sensor, the backscattered portion being backscattered by the eye of the user, and converting the at least partially captured backscattered portion of the laser beam into an electrical signal; and
in at least one recording step, recording signal spikes in the electrical signal which in particular exceed a definable threshold value, together with scan coordinates of the MEMS micromirror system, which are associated with the signal spikes, to detect a pupil of the eye of the user.

13. The pupil detection method according to claim 12, wherein, in at least one contour ascertainment step, at least a portion of a pupil contour of the pupil of the eye of the user is ascertained from a plurality of first signal spikes in different scan rows of the scanned laser beam and/or from a plurality of last signal spikes in different scan rows of the scanned laser beam.

14. The pupil detection method according to claim 13, wherein, in at least one pupil ascertainment step, a pupil shape and/or a pupil position of the pupil of the eye of the user is ascertained from an approximately half pupil contour or from a full-periphery pupil contour.

15. The pupil detection method according to claim 12, wherein the signal spikes recorded in the recording step correspond to speckles which are generated by backscattering of the laser beam, which is applied in the scanning step, at a retina of the user eye.

16. The pupil detection method according to claim 12, wherein, in the recording step, the electrical signal is monitored, using a Schmitt trigger, with respect to whether a threshold value is exceeded, and an interrupt is triggered when the threshold value is exceeded, the interrupt triggering a query of the current scan coordinates of a position of the micromirror system assumed at a time point of the interrupt, using a digital processor.

17. The pupil detection method according to claim 16, wherein, in at least one learning step, the threshold value is iteratively adjusted, with each new frame generated by the scanned laser beam, until a number of signal spikes per frame falls below a definable maximum number.

18. The pupil detection method according to claim 12, wherein, in the scanning step, the laser beam is scanned in alternating directions, including sinusoidally, at least over a majority of the visible portion of the eye of the user.

19. The pupil detection method of claim 18, wherein, at least one electronic element which is provided for capturing the signal spikes in the electrical signal, including a Schmitt trigger and/or a photodiode of the LFI sensor, is deactivated, after a first signal spike has been captured in a scan row of the laser beam scanned in alternating directions, for a remainder of the scan row, and is reset and/or reactivated only once a transition to a subsequent scan row occurs.

20. The pupil detection method according to claim 12, wherein the electrical signal is a voltage signal and, in the conversion of the portion of the laser beam that is backscattered into the LFI sensor into the electrical signal in the capturing step, a photocurrent of a photodiode of the LFI sensor is converted into a voltage signal proportional to the photocurrent, using a transimpedance amplifier.

21. A pupil detection device for a pair of smart glasses, comprising:
at least one projector unit which includes at least one laser feedback interferometry (LFI) sensor and which is configured to scan an infrared laser beam over at least a majority of a visible portion of an eye of a user, using a microelectromechanical system (MEMS) micromirror system of the projector unit, and which is configured at least to capture using the LFI sensor, at least partially, a backscattered portion of the laser beam, the backscattered portion being backscattered by the eye of the user, and which is configured to convert the backscattered portion into an electrical signal; and
an electronic unit configured to recognize signal spikes in the electrical signal which exceed a definable threshold value and to record the signal spikes together with scan coordinates of the MEMS micromirror system which are associated with the signal spikes, to detect a pupil of the eye of the user.

22. A pair of smart glasses, comprising:
a pupil detection device, including:
at least one projector unit which includes at least one laser feedback interferometry (LFI) sensor and which is configured to scan an infrared laser beam over at least a majority of a visible portion of an eye of a user, using a microelectromechanical system (MEMS) micromirror system of the projector unit, and which is configured at least to capture using the LFI sensor, at least partially, a backscattered portion of the laser beam, the backscattered portion being backscattered by the eye of the user, and which is configured to convert the backscattered portion into an electrical signal, and
an electronic unit configured to recognize signal spikes in the electrical signal which exceed a definable threshold value and to record the signal spikes together with scan coordinates of the MEMS micromirror system which are associated with the signal spikes, to detect a pupil of the eye of the user.
